(19) United States
(12) Patent Application Publication
(10) Pub. No.: US 2025/0242344 A1
(43) Pub. Date: Jul. 31, 2025

(54) ASSAY READER

(71) Applicant: mPOD Inc., New York, NY (US)

(72) Inventors: Jeffrey LY, New York, NY (US); Daniel Aaron WISNER, New York, NY (US); Canh LY, New York, NY (US); Mark BOBROW, New York, NY (US); Ruihong WANG, New York, NY (US); Karolina WIESIOLEK, New York, NY (US); Julie WILKINSON, New York, NY (US); Cindy PRYOR, New York, NY (US)

(73) Assignee: mPOD Inc., New York, NY (US)

(21) Appl. No.: 19/111,004

(22) PCT Filed: Sep. 12, 2023
(86) PCT No.: PCT/US2023/032569

§ 371 Date: Mar. 12, 2025

Related U.S. Application Data
(60) Provisional application No. 63/489,998, filed on Mar. 13, 2023.
(60) Provisional application No. 63/405,748, filed on Sep. 12, 2022.

(51) Int. Cl. B01L 3/00; G01N 33/543

(52) U.S. Cl. CPC B01L 3/502715; B01L 3/5023; G01N 33/54388; B01L 2200/025; B01L 2300/021; B01L 2300/0663; B01L 2300/069; B01L 2300/0825

(57) ABSTRACT

Various embodiments described herein allow standardization of test reporting, which in turn increases confidence in lateral flow device testing, reduces user error and increases overall accuracy in POCT results reading in any resource-limited setting for medical diagnostic use. Often the computer readable code comprises a QR code. Also often the QR code is associated with a key specific to the device such that the output data can be securely associated with the specific device and only read with reference to the key.

### RELATED APPLICATIONS

This application claims the benefit of U.S. Provisional Patent Application No. 63/405,748, filed Sep. 12, 2022, and U.S. Provisional Patent Application No. 63/489,998, filed Mar. 13, 2023, both of which are incorporated by reference herein in their entirety.

### STATEMENT REGARDING FEDERALLY SPONSORED RESEARCH OR DEVELOPMENT

This application was partially supported by The National Institutes of Health under Grant 1R44DE030842-01.

### BACKGROUND

Lateral flow assay testing devices rely primarily on human evaluation and comparison of a colored control strip versus an indication strip or line. Other versions of lateral flow testing applications may include color changes or the appearance of color at a portion or all of the test strip when the fluid being tested includes a minimal concentration of the substance being tested, such as a virus, a bacteria, an enzyme, or a chemical or biological compound.

Traditional testing devices are subject to human error in use by improper sample application and human error in interpretation when the test indication is weak or minimally present. The presently described devices, systems and methods address these and other needs in the art.

### SUMMARY

Various embodiments of the present disclosure are directed to systems and methods of recording and evaluating lateral flow assay results from a test strip and digitally reports such results in a compliant manner while authenticating the manner and subject being tested utilizing a dedicated reader device.

According to presently described embodiments, a diagnostic assay reading device is provided comprising: a housing configured to receive a rapid point of care test (POCT) for testing a sample from a subject and adapted to be of a size that it is handheld; a receiving bay configured for positioning the POCT within the housing; optionally a sample introduction port, an illumination source, a detector, a processor and a data/function interface, wherein the POCT is positioned in a receiving bay within the housing, wherein the illumination source and the detector are positioned in optical communication with the receiving bay and with the test area and, if present, the control area of the POCT, wherein the processor is configured to receive and process images collected by the detector, wherein the processing is provided to reduce noise and interference and interpret received signals captured against set criteria to produce output data, and wherein the data interface is transmits or permit transmission of the output data from the diagnostic assay reading device in the form of a graphical user interface and/or a data link.

According to frequently included embodiments, the device further comprises a sample introduction port, the port defining a fluid pathway extending from an external opening to a sample application area of the POCT.

Also according to frequently included embodiments, the device further comprises a sensor adapted to identify or authenticate that a subject using the device is the same subject that provides the sample. According to related frequently included embodiments, the processor is adapted to incorporate the identification or authentication of the subject in the output data. Often in such embodiments, the sensor is adapted to face externally from the device, wherein the sensor comprises a CMOS device and is adapted to capture or log the process of obtaining the sample from the subject and the application of the sample to the POCT.

Also according to frequently included embodiments, the output data is comprised in a computer readable code. Often the computer readable code comprises a QR code. Also often the QR code is associated with a key specific to the device such that the output data can be securely associated with the specific device and only read with reference to the key.

Also according to frequently included embodiments, the POCT is a lateral flow test strip or a vertical flow test device. Often the lateral flow test strip is a nitrocellulose based lateral flow test strip.

Also according to frequently included embodiments, methods of conducting a diagnostic assay are provided comprising: introducing a POCT to the device described herein; introducing a sample containing or suspected of containing an analyte to a sample application area of the POCT; illuminating a test area and/or control area of the POCT using the illumination source; detecting an image signal provided by a label bound or associated with the analyte using the detector in the test and/or control areas; receiving and processing the image signal to reduce noise and background and to determine an attribute of the signal related to signal intensity or power; determining whether a positive or negative signal is present on the POCT associated with the analyte based on the attribute; and generating output data embodying the determination of the positive or negative signal.

Also according to frequently included embodiments, the device further comprises a sample introduction port and the sample is introduced to the device after the POCT is introduced to the device.

Also according to frequently included embodiments, the method further comprises confirming, in an automated manner using a sensor of the device, that the subject providing the sample is the subject using the device. Often the sensor is adapted to face externally from the device, wherein the sensor comprises a CMOS device and is adapted to capture or log the process of obtaining the sample from the subject and the application of the sample to the POCT.

Also according to frequently included embodiments, the method further comprises providing the output data is comprised in a computer readable code. Often the computer readable code comprises a QR code. Also often, the QR code is associated with a key specific to the device such that the output data can be securely associated with the specific device and only read with reference to the key.

Also according to frequently included embodiments, the method further comprises logging one or more of: the time the sample is obtained from the subject, the time the sample is introduced to the POCT; and/or the time that the sample has been in contact with the POCT prior to illumination by the illumination source.

In one example embodiment of the present disclosure, a diagnostic assay reading device comprises: a housing configured to receive a rapid point of care test (POCT); a receiving bay configured for positioning the POCT within the housing; optionally a sample introduction port, an illumination source, a detector, a processor and a data/function interface, wherein the POCT is positioned in a receiving bay within the housing. The illumination source and the detector are positioned in optical communication with the receiving bay and with the test area and, if present, the control area of the POCT. The processor is configured to receive and process images collected by the detector, wherein the processing is provided to reduce noise and interference (e.g., via image stacking and signal processing utilizing gaussian smoothing and least squares) and interpret received signals captured against set criteria related to signal position, signal intensity, relative background intensity, and background intensity artifacts to produce output data. The data interface is provided to transmit or permit transmission of the output data from the automated assay reader to, e.g., the subject, a healthcare practitioner, an insurer or another. The data interface may be in the form of a graphical user interface and/or may be a data link.

In certain embodiments, the detector comprises a CMOS sensor, including a wide band CMOS sensor and/or a wavelength filtered CMOS sensor. In certain other embodiments, the detector comprises a photodiode. In certain embodiments the detector is a reflected signal capture device.

In certain embodiments, a machine learning algorithm and a signal interpretation algorithm are provided and used in the processor in connection with the received images to determine if the image data meets one or more predetermined threshold for determining whether the analyte is present or absent from the POCT or if the test is invalid based on the nature of the POCT, the type of target analyte and/or the imaging conditions. The determination is then recorded to an output data set; and the data interface is utilized to transmit or permit transmission of the output data from the device.

In frequently included embodiments the devices described herein are handheld devices. For example, in certain embodiments the handheld device fits within a volume of 3″ height×5″ length×2″ width. In certain embodiments, the handheld device fits within a volume of 5″ height×5″ length×5″ width. In certain embodiments, the handheld device fits within a volume of 2″ height×5″ length×4″ width. In certain embodiments, the handheld device fits within a volume of 4″ height×4″ length×1″ width. In certain embodiments, the handheld device fits within a volume of 3″ height×5″ length×1″ width. In certain embodiments, the handheld device fits within a volume of 3″ height×6″ length×2″ width.

The POCT may be any of a variety of known POCT devices. Most commonly the POCT is a lateral flow test strip of the type that are commercially available. Exemplary POCTs contemplated herein also include lateral flow tests using non-nitrocellulose matrix materials such as cellulose of other natural polymer or other synthetic polymer matrices provided that they provide a pathway for bibulous and non-bibulous flow of the analyte to a test and/or control areas and are capable of generating a result that can be visually observed and detected by the detectors described herein. Exemplary labels that are detectable using the devices and systems of the present disclosure in connection with the POCTs contemplated herein often include latex beads, colloidal gold particles, fluorescent labels including Qdots and other fluorescent moieties, luminescent such as chemiluminescent labels, and others of the sort. The present devices, systems and methods are used to detect signals generated from analytes bound to any one or more of these latex beads, colloidal gold particles, fluorescent labels including Qdots and other fluorescent moieties, and/or luminescent such as chemiluminescent labels.

In some further still embodiments of the present disclosure multiple reflected images are collected and stacked. The images are stacked and processed to reduce noise and interference, as well as identify any apparitions.

In certain embodiments, of the present disclosure, methods of verifying and reporting a lateral flow assay test (e.g., to a health care provider, insurer, etc.) are provided comprising: introducing a rapid point of care test (POCT) having a test area and optionally a control area to an automated assay reader defined by a housing, and having a receiving bay, optionally a sample introduction port, an illumination source, a detector, a processor and a data/function interface, wherein the POCT is positioned in a receiving bay within the housing. The illumination source and the detector are positioned in optical communication with the receiving bay and with the test area and, if present, the control area of the POCT. A sample obtained from a subject having or suspected of containing an analyte is introduced to the POCT before introduction the automated lateral flow assay testing device. When the sample is introduced to the POCT after being positioned in the automated assay reader, the sample is introduced via a sample introduction port, the port defining a fluid pathway extending from an external opening to a sample application area of the POCT. The processor is configured to receive and process images collected by the detector, wherein the processing is provided to reduce noise and interference and interpret received signals captured against set criteria related to signal position, signal intensity, relative background intensity, and background intensity artifacts to produce output data. In the operation of exemplary embodiment of the method the illumination source illuminates the test and control areas of the POCT after a predetermined time period has passed after sample introduction, the detector captures an image of the illuminated area and the processor processes the image to provide for a determination of whether the analyte is present in the subject, absent from the subject, or whether the test has failed. In practice two or more images are often obtained by the detector and processed by the processor. Often the images are direct images. Also often the images are fluorescent or luminescent images. The type of illumination source and wavelength of the illumination source and detection wavelengths are adjusted based on the type of images to be obtained and processed, e.g., direct, fluorescent or luminescent imaging. The data interface is provided to transmit or permit transmission of the output data from the automated assay reader to, e.g., the subject, a healthcare practitioner, an insurer or another. The data interface may be in the form of a graphical user interface and/or may be a data link. According to further aspects of the present methods, the results of the test are provided via an encrypted file, wherein the encrypted file is a QR code unique to the individual test performed and the QR code encodes the results of the test.

In certain embodiments the present disclosure, the device may comprise one or more of the following features, in any combination from a single feature, to combination of two or more features, to comprising all of the following features: the illumination source illuminates a portion of the POCT within a wide band visual spectrum; the illumination source illuminates a portion of the POCT within a narrow-band wavelength for fluorescent excitation; the illumination source illuminates a portion of the POCT within the wide band visual spectrum and within the narrow-band wavelength for fluorescent excitation; the detector comprises a wide band CMOS sensor; the detector comprises a wavelength filtered CMOS sensor; the detector comprises at least one of a wide band CMOS sensor and a wavelength filtered CMOS sensor; the processor is configured to capture at least one reflected image within the wide band visual spectrum and one image within the narrow-band wavelength for fluorescent excitation; the processor is configured to receive at least two or more images within the wide band visual spectrum and at least two or more images within the narrow-band wavelength for fluorescent excitation; the processor is configured to receive and combine multiple detected image signals and stack the captured image signals to facilitate noise reduction; the processor is configured to standardize the captured reflected signals; wherein the processor is configured to standardize the captured reflected signals and further process the signals using at least one of a machine learning signal processing algorithm and a signal interpretation algorithm to determine whether the captured signal meets a predetermined threshold and record the determination as output data; the device wherein the output data is configured for transmission from the device via at least one of a graphical user interface; a QR code generated for each set of output data; a WiFi, Bluetooth, or cellular connection; or via direct network connection; and the output data is stored locally at the processor.

These and other embodiments, features, and advantages will become apparent to those skilled in the art when taken with reference to the following more detailed description of various exemplary embodiments of the present disclosure in conjunction with the accompanying drawings.

### BRIEF DESCRIPTION OF THE DRAWINGS

Various embodiments are disclosed in the following detailed description and accompanying drawings. The skilled person in the art will understand that the drawings, described below, are for illustration purposes only.

FIG. 1 illustrates a diagrammatic representation of a computing device on which the embodiments of the present disclosure can be implemented.

FIG. 2A illustrates an exploded view of an exemplary assay reader according to embodiments herein.

FIG. 2B illustrates the top, transparent view of the assay reader in FIG. 2A.

FIG. 2C illustrates a perspective, transparent view of an assay reader with its components fully assembled.

FIG. 2D illustrates the top, transparent view of the assay reader in FIG. 2B.

FIG. 3 illustrates a diagrammatic representation of a networked computer environment and system 900 on which the embodiments of the present disclosure can be implemented.

FIG. 4 illustrates a flow chart depicting an example embodiment of a method of the present disclosure.

FIG. 5 illustrates a graphic comparison of test results including those obtained using example embodiments of the present disclosure.

FIG. 6 illustrates a comparison of test results including those obtained using example embodiments of the present disclosure.

FIG. 7A illustrates a comparison of test results including those obtained using example embodiments of the present disclosure.

FIG. 7B illustrates another comparison of test results including those obtained using example embodiments of the present disclosure

FIG. 8A illustrates test result indications on common POCTs.

FIG. 8B illustrates test result indications on other common POCTs.

FIG. 8C illustrates test result indications on yet other common POCTs.

### DETAILED DESCRIPTION

For clarity of disclosure, and not by way of limitation, the detailed description of the disclosure is divided into the subsections that follow.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, “faint positive” refers to a result of a test involving a POCT where the result is only faintly visible or perhaps not even discernible via the naked human eye. In this regard, the sensitivity of concentration levels of analyte detectable by the present devices and systems are provided at levels below that is detectable via the naked human eye. Exemplary analyte concentrations embodied within the meaning of “faint positive” are described herein and are merely exemplary based on the type of label used in the specific example and type of POCT. Detection of analyte concentrations at or below the levels identified by “faint positive” in specific examples herein by the presently described devices and systems are contemplated herein.

As used herein, “lateral flow test strip” refers to one exemplary form of rapid point of care test (POCT). Other POCTs are contemplated for use by the devices and systems of the present disclosure according to the disclosed methods. Most commonly the POCT is a lateral flow test strip of the type that are commercially available. Exemplary POCTs contemplated herein also include lateral flow tests using non-nitrocellulose matrix materials such as cellulose of other natural polymer or other synthetic polymer matrices provided that they provide a pathway for bibulous and non-bibulous flow of the analyte to a test and/or control areas and are capable of generating a result that can be visually observed and detected by the detectors described herein. Exemplary labels that are detectable using the devices and systems of the present disclosure in connection with the POCTs contemplated herein often include latex beads, colloidal gold particles, fluorescent labels including Qdots and other fluorescent moieties, luminescent such as chemiluminescent labels, and others of the sort. The present devices, systems and methods are used to detect signals generated from analytes bound to any one or more of these latex beads, colloidal gold particles, fluorescent labels including Qdots and other fluorescent moieties, and/or luminescent such as chemiluminescent labels.

As used herein, “wide band visual spectrum” is not intended to be limiting and generally refers to illumination that provides a reflected signal from a target label that is primarily detectable in the non-fluorescent and non-luminescent visual spectra even if it is accompanied by fluorescent and/or non-luminescent visual signals.

As used herein, “narrow-band wavelength” is not intended to be limiting and generally refers to illumination that provides excites and provides a fluorescent or luminescent signal from a target label that is primarily detectable in the fluorescent and luminescent visual spectra even if it is accompanied by a non-fluorescent and/or non-luminescent visual signal. For example, for Europium dyes and Qdots excitation is provided in the sub-400 nm range and Europium's peak excitation is 365 nm, which are below the visual spectrum in the UV spectrum. Other narrow-band wavelength excitations by the described illumination sources are contemplated herein that correspond to all commercially known fluorescent labels that are used or can be used in POCTs.

As used herein, “reduce noise and interference” refers to a manner of obtaining a more accurate indication of images of the target label on the POCT. Often this process is performed using image stacking and signal processing utilizing gaussian smoothing and least squares, but other methods are contemplated. According to a frequently included aspect of the presently contemplated embodiments, the processing of the images to reduce noise and interference is provided in a manner that erases the background noise and any non-related streaking and other artifacts so that the true signal from the target label is provided as a distinct and discernable peak. The presently contemplated algorithms provide for this to be performed in an automated manner in the device based on the type and nature of the POCT, the target analyte and target label type.

As used herein, “set criteria” is primarily related to signal position, signal intensity, relative background intensity, and background intensity artifacts. Based on the type of POCT, the base material of the underlying matrix material, the analyte to be examined and the type of labels used in the POCT, the set criteria adjust to the location of the test and control areas and to identify and create the ideal signal intensity, relative background intensity, and reduce background intensity artifacts to produce output data.

As used herein, the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues comprise an aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

“Fluid sample” refers to a material suspected of containing the analyte(s) of interest, which material has sufficient fluidity to flow through an immunoassay device in accordance herewith. The fluid sample can be used as obtained directly from the source or following a pretreatment so as to modify its character. Such samples can include human, animal or man-made samples. The sample can be prepared in any convenient medium which does not interfere with the assay. Typically, the sample is an aqueous solution or biological fluid as described in more detail below.

The fluid sample can be derived from any source, such as a physiological fluid, including blood, serum, plasma, saliva, sputum, ocular lens fluid, sweat, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Herein, fluid homogenates of cellular tissues such as, for example, hair, skin and nail scrapings, meat extracts and skins of fruits and nuts are also considered biological fluids. Pretreatment may involve preparing plasma from blood, diluting viscous fluids, and the like. Methods of treatment can involve filtration, distillation, separation, concentration, inactivation of interfering components, and the addition of reagents. Besides physiological fluids, other samples can be used such as water, food products, soil extracts, and the like for the performance of industrial, environmental, or food production assays as well as diagnostic assays. In addition, a solid material suspected of containing the analyte can be used as the test sample once it is modified to form a liquid medium or to release the analyte. The selection and pretreatment of biological, industrial, and environmental samples prior to testing is well known in the art and need not be described further.

“Analyte” refers to the compound or composition to be detected or measured and which has at least one epitope or binding site. The analyte can be any substance for which there exists a naturally occurring analyte specific binding member or for which an analyte-specific binding member can be prepared. e.g., carbohydrate and lectin, hormone and receptor, complementary nucleic acids, and the like. Further, possible analytes include virtually any compound, composition, aggregation, or other substance which may be immunologically detected. That is, the analyte, or portion thereof, will be antigenic or haptenic having at least one determinant site, or will be a member of a naturally occurring binding pair.

Analytes include, but are not limited to, toxins, organic compounds, proteins, peptides, microorganisms, bacteria, viruses, amino acids, nucleic acids, carbohydrates, hormones, steroids, vitamins, drugs (including those administered for therapeutic purposes as well as those administered for illicit purposes), pollutants, pesticides, and metabolites of or antibodies to any of the above substances. The term analyte also includes any antigenic substances, haptens, antibodies, macromolecules, and combinations thereof. A non-exhaustive list of exemplary analytes is set forth in U.S. Pat. No. 4,366,241, at column 19, line 7 through column 26, line 42, the disclosure of which is incorporated herein by reference. Further descriptions and listings of representative analytes are found in U.S. Pat. Nos. 4,299,916; 4,275,149; and 4,806,311, all incorporated herein by reference.

Embodiments of the present disclosure are directed to systems and methods of reading a POCT using a networked, dedicated reading device or reader, configured to apply clinical AI and deep learning by leveraging TinyML to drive the intelligence behind the analysis of clinically relevant images in a medical application. Example embodiments of the present disclosure are applicable to manufacturer and healthcare provider demands for accurate test strip results, as highlighted most recently by the COVID-19 pandemic for medical application including but not limited to accurate and standardized reading of lateral flow tests.

Various embodiments described herein allow standardization of test reporting, which in turn increases confidence in lateral flow device testing, reduces user error and increases overall accuracy of an AI-driven lateral flow device testing program leveraging TinyML (otherwise known as TensorFlow Lite for Microcontrollers) in any resource-limited setting for medical diagnostic use. Rapid antigen lateral flow devices allow for testing of large asymptomatic or symptomatic populations for SARS-COV-2, alongside mass testing programs for a variety of other medical indications. Lateral flow devices have a different sensitivity range to viral load, and can be used to self-test, when compared to real-time quantitative PCR testing. When used correctly, these tests have the potential to reduce disease transmission, however self-testing presents challenges for the lay user to interpret, especially at low viral loads, and significant variability is observed in device interpretation.

The presently contemplated devices can be used in connection with POCTs for a variety of analytes in addition to SARS-COV-2. For example, analytes including but not limited to the following may be target analytes that can be evaluated in connection with POCTs of the present disclosure: FluA, FluB, target analytes in drugs of abuse, women's health, fertility, cardiovascular health, cancer screening, etc. The present devices and systems are provided in an analyte agnostic form with the limitations and aspects described herein concerning the fact that the POCT must generate a detectable result.

In various example embodiments of the present disclosure, the reader system comprises a software application that is available as a smartphone app and web application that uses an end-to-end deep-learning cloud-based algorithm to automate the reading and analysis of different diagnostic lateral flow tests in under a second. The reader instructs the end user to administer a lateral flow test and interpret the test result, whilst also enabling the quick detection and presence of otherwise undetected low viral loads of various target viruses, such as SARS-COV-2. Rapid retraining of the system allows for endless future indications including multiplex lateral flow devices, and applications across the infectious diseases, cancer, and broader medical and surgical specialties. The system then provides a digital certificate that embodies the test result, and the associated health status, while providing immediate governmental advice on the following instructions, for example, the device can provide meaningful population-level trends to allow health policies. The system is platform agnostic concerning the type of test device utilized (e.g., lateral flow and other test devices) as long as the device provides a visually detectable or fluorescent signal, and provides test interpretation that is fast, accurate, and secure, ensuring that results are confidential and cannot be tampered with.

An exemplary device of the present disclosure may in certain embodiments include a reader configured to provide an end-to-end deep learning, cloud-based API that can read POCT results, which reduces risk of human error, by enabling visual interpretation that spans beyond the human visual spectrum. Readers described herein are adapted to provide results to the user in far faster times then prior-art test strip readers, such as returning results in under 1 minute, under 45 seconds, under 30 seconds, under 20 seconds, under 10 seconds, under 5 seconds, under 2 seconds, and/or under 1 second. Moreover, readers of the present disclosure may be used in any setting globally alone or in connection with the use of a smartphone or other networked device and can be trained to read any quantitative and multiplexed assay POCT.

As contemplated herein the present devices and systems can be utilized in POCTs that are adapted for detection of two or more analytes in a multiplexed assay. For example, in such an embodiment each of the two or more different analytes may become bound or associated with a label that has different and/or distinguishable detection characteristics and the device is adapted to simultaneously detect the two or more analytes, often in different images in the examples where a different excitation/illumination wavelength is necessary to cause the generation of a detectable signal. In certain related embodiments the POCT includes two or more different fluorescent and/or chemiluminescent labels and the device utilized illumination in the narrow band necessary to generate a distinct signal from each of the two or more different fluorescent and/or chemiluminescent labels and the detector is configured to receive and process images including the two or more different fluorescent and/or chemiluminescent labels.

According to embodiments described herein the presently described devices and systems are capable of detecting positive results of a POCT that would otherwise often be mis-determined as a negative test result when viewed by the naked human eye. As such the present devices and systems provide a sensitivity well beyond that of the naked human eye, which increased the utility and sensitivity of the POCT tests often in certain circumstances to the level of equivalent molecular testing. In this sense, the presently described devices can detect faint positive.

### Hardware

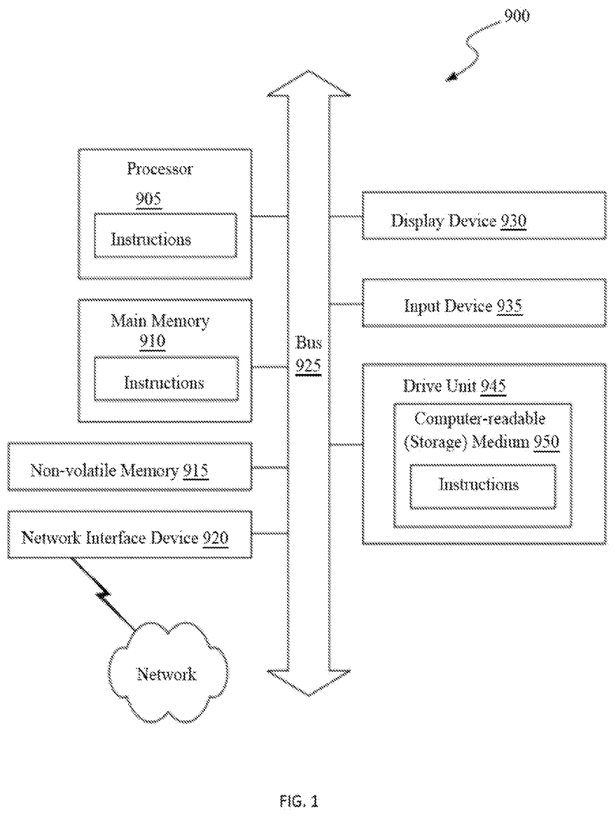

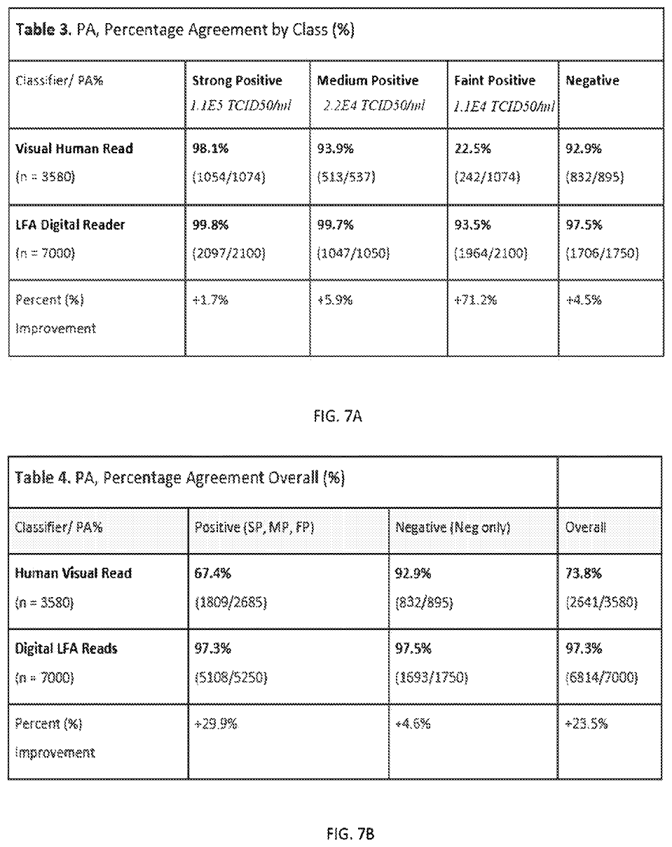

FIG. 1 shows a diagrammatic representation of a computer system 900 on which the embodiments of the present disclosure can be further implemented. Computer system 900 may be a remote device, appliance, server or other computing device described it the computing environment of FIG. 7. The computer system 900 may be in the form of a mobile device, a tablet computing device, a laptop device, a work station, or a dedicated device configured to implement the steps and functionality described it the present disclosure. The computer system 900 generally includes a processor 905, main memory 910, non-volatile memory 915, and a network interface device 920. Various common components (e.g., cache memory) are omitted for illustrative simplicity but would be known to a person of ordinary skill in the art. The computer system 900 is intended to illustrate a hardware device on which any of the components and methods described above can be implemented. The computer system 900 can be of any applicable known or convenient type. The components of the computer system 900 can be coupled together via a bus 925 or through some other known or convenient device.

The processor 905 may be, for example, a conventional microprocessor such as an Intel Pentium microprocessor or Motorola power PC microprocessor, or other commercially available microprocessors. One of skill in the relevant art will recognize that the terms “computer system-readable (storage) medium” or “computer-readable (storage) medium” include any type of device that is accessible by the processor.

The main memory 910 is coupled to the processor 905 by, for example, a bus 925 such as a PCI bus, SCSI bus, or the like. The main memory 910 can include, by way of example but not limitation, random access memory (RAM), such as dynamic RAM (DRAM) and static RAM (SRAM). The main memory 910 can be local, remote, or distributed.

The bus 925 also couples the processor 905 to the non-volatile memory 915 and drive unit 945. The non-volatile memory 915 is often a magnetic floppy or hard disk, a magnetic-optical disk, an optical disk, a read-only memory (ROM), such as a CD-ROM, EPROM, or EEPROM, a magnetic or optical card, an SD card, or another form of storage for large amounts of data. Some of this data is often written, by a direct memory access process, into memory during execution of software in the computer system 900. The non-volatile memory 915 can be local, remote, or distributed. The non-volatile memory can be optional because systems can be created with all applicable data available in memory. A typical computer system will usually include at least a processor, memory, and a device (e.g., a bus) coupling the memory to the processor.

Software is typically stored in the non-volatile memory 915 and/or the drive unit 945. Indeed, for large programs, it may not even be possible to store the entire program in the memory. Nevertheless, it should be understood that for software to run, it is moved, if necessary, to a computer readable location appropriate for processing, and for illustrative purposes, that location is referred to as the main memory 910 in this disclosure. Even when software is moved to the memory for execution, the processor will typically make use of hardware registers to store values associated with the software and the local cache. Ideally, this use serves to speed up execution. As used herein, a software program is assumed to be stored at any known or convenient location (from non-volatile storage to hardware registers) when the software program is referred to as “implemented in a computer-readable medium”. A processor is considered to be “configured to execute a program” when at least one value associated with the program is stored in a register readable by the processor.

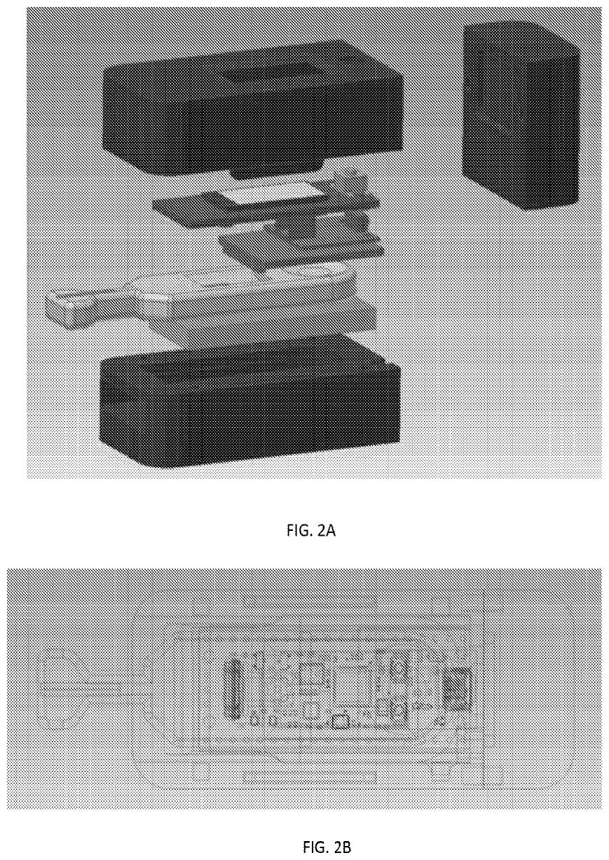

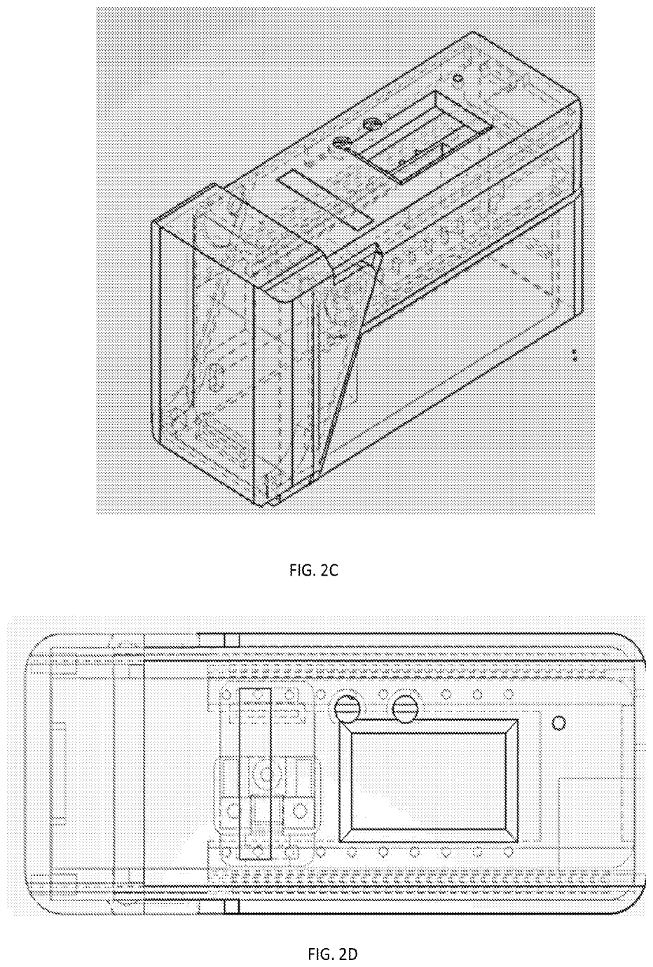

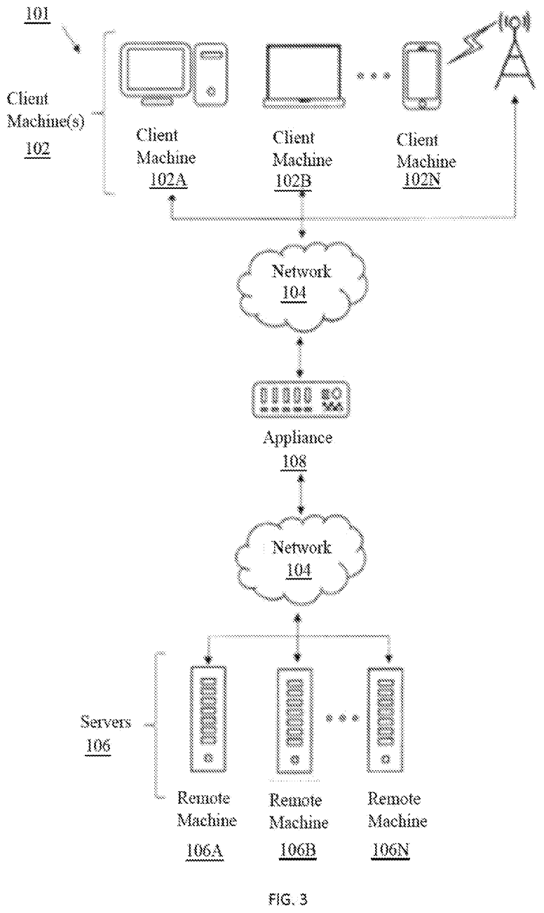

The bus 925 also couples the processor to the network interface device 920. The interface can include one or more of a modem or network interface. It will be appreciated that a modem or network interface can be considered to be part of the computer system 900. The interface can include an analog modem, ISDN modem, cable modem, token ring interface, satellite transmission interface (e.g., “direct PC”), or other interfaces for coupling a computer system to other computer systems. The interface can include one or more input and/or output devices 935. The I/O devices can include, by way of example but not limitation, a keyboard, a mouse or other pointing device, disk drives, printers, a scanner, speakers, DVD/CD-ROM drives, disk drives, and other input and/or output devices, including a display device. The display device 930 can include, by way of example but not limitation, a cathode ray tube (CRT), a liquid crystal display (LCD), an LED display, a projected display (such as a heads-up display device), a touchscreen or some other applicable known or convenient display device. The display device 930 can be used to display text and graphics. For simplicity, it is assumed that controllers of any component not depicted in the example of FIG. 2A-C reside in the interface.

In operation, the computer system 900 can be controlled by operating system software that includes a file management system, such as a disk operating system. One example of operating system software with associated file management system software is the Windows® family of operating systems from Microsoft Corporation and their associated file management systems. Another example of operating system software with its associated file management system software is the Linux operating system and its associated file management system. The file management system is typically stored in the non-volatile memory 915 and/or drive unit 945 and causes the processor to execute the various acts required by the operating system to input and output data and to store data in the memory, including storing files on the non-volatile memory 915 and/or drive unit 945.

In a further embodiment of the present disclosure, a reader may be configured to leverage the intelligence of the TinyML model to keep hardware requirements simple and low-cost including low-cost microcontrollers, particularly those leveraging Arm Cortex-M Series architecture and detection hardware such as low-resolution CMOS camera modules as well as other optical detection systems. Various embodiments of the present disclosure may use off-the-shelf hardware system that serve as example of microcontrollers the reader system will leverage to deploy its TinyML Model, including:
Arduino Nano 33 BLE Sense
SparkFun Edge
STM32F746 Discovery kit
Adafruit EdgeBadge
Adafruit TensorFlow Lite for Microcontrollers Kit
Arducam Pico4ML
Adafruit Circuit Playground Bluefruit
Espressif ESP32-DevKitC
Espressif ESP-EYE
Wio Terminal: ATSAMD51
Himax WE-I Plus EVB Endpoint AI Development Board
Synopsys DesignWare ARC EM Software Development Platform
Sony Spresense

In one exemplary embodiment, the reader comprises various low-cost, mass producible hardware, encapsulated in a bespoke housing (produced, e.g., by injection molding) designed to standardize the position and lighting of the LFA being analyzed. In certain frequently included embodiments, the housing incorporating all aspects of the present devices and systems is handheld or of a size that can be handheld. The ability to have a TinyML based model drive intelligence that can run off low-cost, mass producible hardware provides for scalability, making hundreds of millions of reader units commercially viable, opening up practical accessibility and practicality of low-cost LFA diagnostics possible anywhere globally, regardless of infrastructure.

With reference to FIG. 2A-C, an example embodiment of the present disclosure is provided, comprising: a diagnostic assay reading device, or reader 200; a body 210 configured to receive a POCT 215 and further housing; a receiving bay 220 configured for positioning the POCT 215 within the housing; an illumination source 225 positioned adjacent the receiving bay 220; a detector 230; a processor 250 configured to: capture images collected at the detector 230; process images collected to reduce noise and interference; interpret signals captured against set criteria to produce output data; and a data interface 260 to permit transmission or transmit the output data from the diagnostic assay reading device, or reader 200.

In still a further example embodiment of the diagnostic assay reading device 200 of the present disclosure a device may comprise: a body 210 configured to receive a POCT 215 and further housing; a receiving bay 220 configured for positioning the POCT 215 within the body 210; an illumination source 225 positioned adjacent the receiving bay 220 wherein the illumination source is configured to illuminate a portion of the POCT 215 within at least one of a wide band visual spectrum and within a narrow-band wavelength for fluorescent excitation; a detector 230 comprises at least one of a wide band CMOS sensor 231 and a wavelength filtered CMOS sensor 232; a processor 250 configured to: capture images collected at the detector 230 wherein the images comprise at least two or more images within the wide band visual spectrum, at least two or more images within the narrow-band wavelength for fluorescent excitation, or at least one image within the wide band visual spectrum and at least one from within a narrow-band wavelength for fluorescent excitation; the processor 250 further processes images collected to reduce noise and interference; interpret signals captured against set criteria using at least one of a machine learning algorithm and a signal interpretation algorithm to determine if the captured image data meets a predetermined threshold and records the determination to an output data set; and a data interface 260 to transmit or permit transmission of the output data from the diagnostic assay reading device 200, wherein the interface comprises at least one of a graphical user interface, a QR Code, a WiFi, Bluetooth, or cellular connection, or a direct network connection.

In yet another example embodiment, a method of use of various embodiments of the diagnostic assay reading device 200 of the present disclosure may comprise the steps of: (410) Insert a POCT into the body of the diagnostic assay reading device (420) e.g., within a receiving bay of the body of the device, wherein the receiving bay may be incorporated into a slot receiver, an alligator or clam shell type receiver, or a tray that is at least partially removable from the body and then inserted into the body of the device; (430) the POCT is positioned within the receiving bay in optical communication with an illumination source so that at least the test and control areas of the POCT are illuminated using at least one of a visual spectrum illumination source and a fluorescent dye illumination/excitation source; (440) activating the device after placement of the test strip; (450) illuminating the test strip with one or more of the light/illumination sources comprising at least one of the wide band visual spectrum and within a narrow-band wavelength for fluorescent excitation; (460) capturing with an image from the illuminated POCT in at least one of either the visual spectrum using a CMOS sensor or within the narrowband fluorescent wavelength using a wavelength filtered CMOS sensor; (470) collecting the captured images in a data stack, such that at least one image from the visual spectrum and one image from within the fluorescent band are collected. In some embodiments multiple reflected images are collected and stacked. The images are stacked and processed to reduce noise and interference, as well as identify any apparitions. In certain embodiments the device includes a sample application port such that a sample containing or suspected of containing an analyte can be introduced to the POCT after the POCT is positioned in the bay. In other embodiments, the sample containing or suspected of containing an analyte is introduced to the POCT prior to placing the POCT in the receiving bay.

The processor then takes the stacked images and uses any standardization functions to standardize the images for further processing. For example, illumination intensity and normalizing of overall signal reflected into the sensor so that reading between reader to reader as well as strip to strip can be normalized. In some embodiments standardization is done via squared power of the CMOS signal.

The standardized stack images are then further processed to determine if the reflective signals meet a predetermined criteria to indicate a positive or negative LFA exposure. In one embodiment, the further processing is done through a machine learning algorithm. In yet another embodiment, the further processing is done using a signal processing algorithm. In still further embodiments multiple algorithms are used and compared to achieve a higher level of confidence of the determination of the results of the test.

The determination, whether positive or negative, is save to an output data file. The output data file may contain only the final determination of the test result or may contain one or more of the stacked images processed to make the test determination.

Finally, the output data is configured to be output via a graphic user interface, a QR code, or a network connection, such as a WiFi, NFC, Bluetooth, cellular, or direct network.

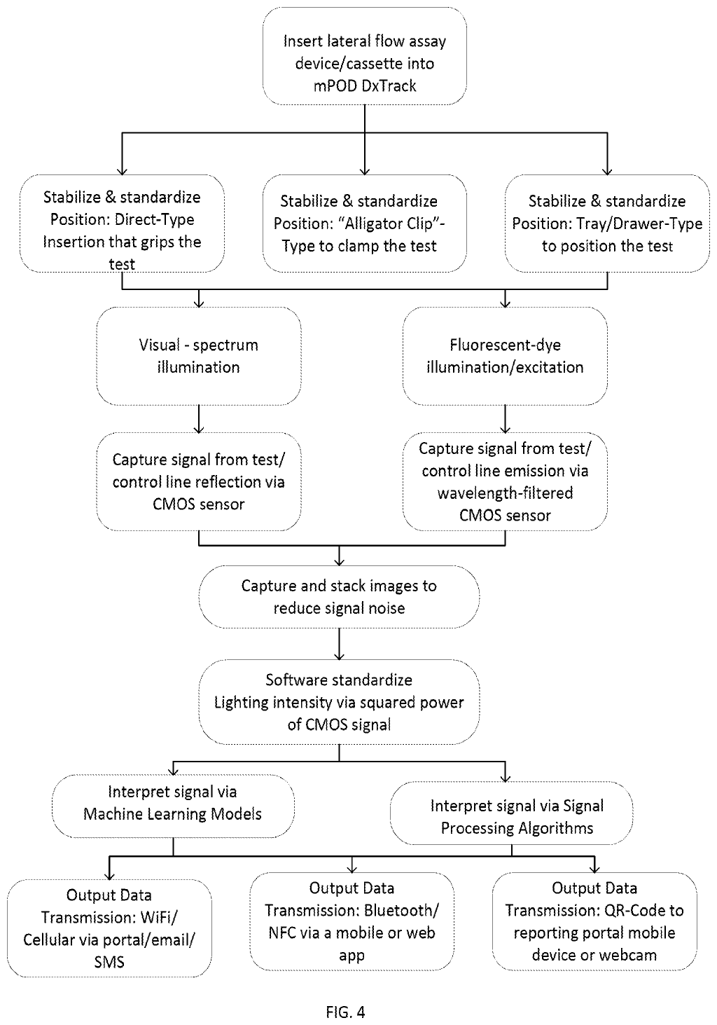

FIG. 4 illustrates an example method of use of an embodiment of the reader disclosed herein.

### Software

In example embodiments of the present disclosure, the reader 200 is driven by a TensorFlow Lite model designed/miniaturized to fit on simple low-power and low-cost microcontrollers with imaging driven by low-cost, low-resolution optics. Advantages of such example embodiments provide a model robust enough to provide >99.9% accuracy while facilitating the operating the constraints of low-cost hardware. Enabling software of the present disclosure may comprise:

### Platform Architecture Encompassing Admin Panel (Frontend and Backend) and User App.

Three roles—super-admin, admin with access to statistics, and user.
Super-admin—user list, management and statistics
Authentication architecture
Terms of use and Privacy Policy documents
GDPR & HIPAA compliance
User data including email, name/surname/location
Record database, search and filter
Test progression chart
UI/UX design with healthcare focus
Cloud-based Application Server
Web App deployment
iOS mobile application deployment (App Store)
Android mobile application deployment (Play Market)

In one example embodiment of the present disclosure, the reader model is developed in TensorFlow/Keras and trained with images collected off the reader hardware suite. This process is iterative and based on collecting closed-loop pipeline where images are fed back to continue improving the model over time across an ever-growing set of user-generated data.

The benefits of embodiments of the reader are outlined under the “SAS” pillars of Simple, Accessible, and Smart. Under the “SAS” pillars, key unique benefits include: simple—accurate, automated and flexible testing; accessible—rapid, easy-to-use, real-world testing; and smart—validated and secure testing with AI-informed population-level insights. These are to assist with—testing, providing digital connectivity; reading, streamlining lateral flow interpretation; and validating, integrating the final crucial step of digital personal identification.

The present disclosure is further described by the following examples. The examples are provided solely to illustrate the invention by reference to specific embodiments. These exemplifications, while illustrating certain specific aspects of the disclosure, do not portray the limitations or circumscribe the scope of the disclosed innovations.

### Example 1

The preliminary data presented here was generated to demonstrate the improvement of POCT interpretation by the various embodiments of the present disclosure, for example, the reader platform over human visual interpretation (HVI) for an over-the-counter (OTC) lateral flow COVID-19 antigen test. As HVI is the basis for clinical performance and FDA authorization for lateral flow devices, this study quantifies the benefit for implementation of the reader for the interpretation of test results to improve test accuracy, sensitivity and precision as well as provide a means for digital reporting test results on a population scale.

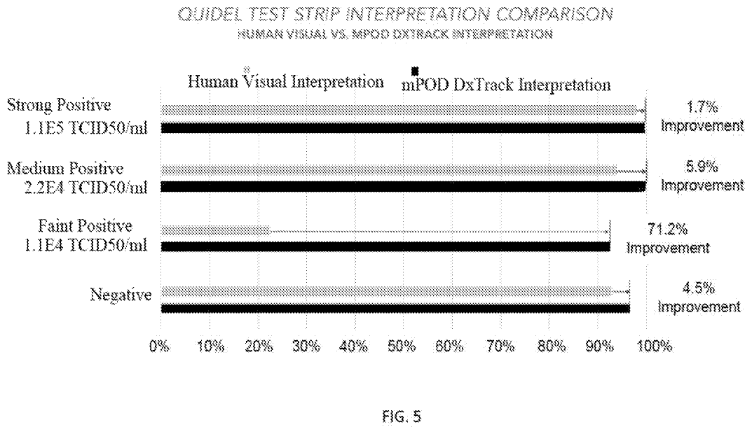

As shown in FIG. 5, When using an example embodiment of the reader platform of the present disclosure, the QuickVue™ At-Home OTC COVID-19 test strip interpretation improved over Visual Human Interpretation on all levels of test strip intensity (SP, MP, FP & Neg), with the greatest improvement (+71.2%) in the Faint Positive (FP) class.

Overall, when compared to Visual Human Interpretation, the example reader improved accuracy by +29.9% for Positive Test strips and 4.6% for Negative Test strips over n=7000 DxTrack reads and n=3580 Human Visual Interpretations.

The reader disclosed herein, increases sensitivity of the QuickVue At-Home OTC COVID-19 test kit.

Quidel's™ current FDA EUA filing estimates a limit-of-detection that is closely equivalent to the Medium Positive [MP] test line intensity conducted in this study. In this Medium Positive class of test line intensity, the example embodiment of the reader provides an improvement (+5.8%)—translating to reducing missed positive results that currently exist simply from misinterpretation by the end-user in the population.

With the support of the BARDA EZ-BAA, the reader may provide nearly a 2× improvement from (1.9E4 TCID50/ml down to 1.1E4 TCID50/ml) in the limit of detection of the QuickVue At-Home OTC COVID-19 Test Kit without any changes to the design or manufacture of the test kits themselves.

Analog-to-Digital Interpretation for Streamlined Reporting of COVID-19 Test Results in at-Home Settings

The example embodiment of the reader disclosed herein demonstrates how an analog (visually interpreted) result from an LFA strip can be reliably digitized with a time-stamp, dramatically improving utility of any POC or OTC LFA test.

In addition to reducing the number of missed positive results at Quidel's current limit of detection and increasing clinical performance of the assay overall, the reader provides objective standardized interpretation to build confidence in current LFA testing platforms.

### Experimental Protocol

Quidel QuickVue At-Home OTC COVID-19 test kits were purchased at local pharmacies and prepared following the provided IFU and modified so that NR-52287 Isolate USA-WA1/2020, Gamma-Irradiated (GIV) SARS-COV-2 Stock was spiked with the following concentrations:

| | |
|---|---|
| Strong Positive [SP] | (1.1E5 |
| | TCID50/ml) |
| *Medium Positive | (2.2E4 |
| [MP] | TCID50/ml) |
| Faint Positive [FP] | (1.1E4TCID50/ml) |
| Negative [Neg] | N/A |

*Utilizing the FDA's publicly released filing, Medium Positive [MP] matches Quidel's current estimated LoD

A total of twenty 20 strips were prepared: 6 Strong Positive, 3 Medium Positive, 6 Faint Positive and 5 Negative. These were used in comparing human visual interpretation vs. the reader platform.

### Visual Human Interpretation

At the 10-minute mark, photos were taken of each strip run. These strips were then uploaded and randomized in Google Form. Each study participant was given 5 minutes to complete the interpretation of all 20 strips as either Positive or Negative with the mindset that “the test result would determine whether or not it was safe to visit a loved one.” 179 individuals participated in this study, generating a 179×20=3580 distinct datapoints to analyze.

### Reader Interpretation

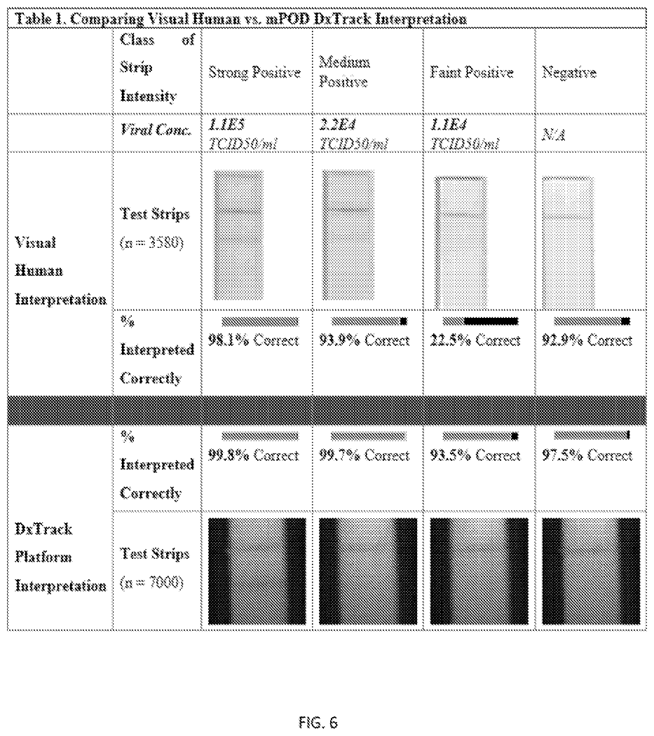

The same 20 strips used for human visual interpretation were then used for interpretation by the reader. Between 10 and 15 mins after the test was started, reader-based images were taken of each test strip. To add variety and authenticity to the data collected, purpose-built hardware was used to randomly vary 1) Horizontal Position, 2) Vertical Position, and 3) Lighting intensity to represent a variety of reading scenarios that could be encountered in the field. Each strip was interrogated 350 times, generating 7,000 distinct datapoints. The images were interpreted in real time by the reader platform with either a Positive or Negative. In total, 350 distinct reads per strip were made in this study, generated a 350×20=7000 distinct datapoints to analyze. As illustrated in FIGS. 6A and 6B, the reader of the present disclosure showed improvement over visual human reads of the test strip in all categories, and notably a 71.2% improvement over faint positive indications.

### Steps

The reader is easily scalable and can be interfaced with any visually read LFA and OTC test available on the market. The commercial target of the reader is to achieve a >99% overall accuracy in interpreting LFA test strips.

Protocol

| | |
|---|---|
| NR-52287 Isolate USA-WA1/2020, | 2.8 × 10E6 TCID50/ml |
| Gamma-Irradiated (GIV) Stock | |
| Quidel claimed LoD | 1.9 × 10E4 TCID50/ml |

Per the FDA filing, 50 ul of “nasal matrix” was used in the analytical testing by Quidel—this is the projected volume that carries the 1.9×10E4 TCID50/ml.

When the swab used to collect 50 ul of “nasal matrix” with 1.9×10E4 TCID50/ml is introduced to the provided 333 ul of Quidel Kit Buffer, this viral protein concentration would dilute the viral content to 2.8×10E3 TCID50/ml. Projecting that 50% of viral protein in the swab is released when introduced in the Quidel Kit Buffer, we are left with an equivalent to 1426 TCID50/ml of GIV in the 333 ul of Quidel Kit Buffer. This will serve as the baseline viral protein load when analyzing prepared strips with varying viral protein loads.

In this study, rather than load a “nasal matrix” from a swab, to ensure reproducible and controlled viral protein load is introduced into the Quidel Kit Buffer each time, a 10 ul dilution of the NR-52287 Isolate USA-WA1/2020, Gamma-Irradiated (GIV) Stock is pipetted into the Quidel Kit Buffer instead.

TABLE 1

| | | | | Run Concentration | Concentration |
|---|---|---|---|---|---|
| | Summary of | Dilution | Dilution | once 10 ul of dilution | of Material that |
| | Concentrations | Concentration | Concentration | is spiked into Quidel | the Swab is |
| Strip | (Dilution of | of Stock | (Genome | Kit Buffer | loaded with |
| Intensity | Stock GIV) | GIV | Equivalents) | (TCID50/ml) | (TCID50/ml) |
| Strong | 1:10 | 2.8 × 10\hat{ }5 | 1.5 × 10\hat{ }8 | 8400 | 1.1 × 10\hat{ }5 |
| Positive (SP) | | TCID50/ml | Genome/ml | | TCID50/ml |
| Medium | 1:50 | 5.6 × 10\hat{ }4 | 3.0 × 10\hat{ }7 | 1680 | 2.2 × 10\hat{ }4 |
| Positive (MP) | | TCID50/ml | Genome/ml | | TCID50/ml |
| Faint | 1:100 | 2.8 × 10\hat{ }4 | 1.5 × 10\hat{ }7 | 840 | 1.1 × 10\hat{ }4 |
| Positive (FP) | | TCID50/ml | Genome/ml | | TCID50/ml |
| | QUIDEL | | | 1426 | 1.9 × 10\hat{ }4 |
| | Estimated LoD | | | | TCID50/ml |

Make dilution of GIV at the above Dilutions in 1×PBS.
Add 10 ul to the 333 ul of Buffer reaction and vortex the tube before spinning down. Incubate for 1 min.
Drop the Strip in for 10 mins
Begin gathering data/validating Reader Performance
Readings were not taken beyond 15 mins.

The reader disclosed herein leverages TFLM and low cost hardware to enable accurate, rapid and objective interpretation of currently available lateral flow assays (LFAs) in less than 10 seconds. LFAs serve as diagnostic tools because they are low-cost and simple to use without specialized skills or equipment. Most recently popularized by COVID-19 rapid antigen tests, LFAs are also used extensively in testing for pregnancy, fertility and women's health issues, disease tracking, STDs, food intolerances, alcohol, drugs along with an extensive array of biomarkers totaling billions of tests sold each year. The reader is applicable to use with any type of visually read lateral flow assay, demonstrating a healthcare use case for TFLM that can directly impact our everyday lives.

The LFA begins with a sample (nasal swab, saliva, urine, blood, etc) loaded at (1) in a sample application area such as a sample application port. Once the sample has flowed to the conjugate zone (2), any existing analyte binds a labeled moiety. Through bibulous and non-bibulous flow, the labeled analyte flows through the test strip substrate to a capture line where it is immobilized at (3). With most LFA tests, two lines indicate a positive result, one line indicates a negative result. Embodied in these two lines are control and test areas. The control area captures labeled moiety, often regardless of whether it is bound to the target analyte and the test area captures labeled moiety that is bound to the target analyte. This explanation is exemplary only as a person of ordinary skill in the art would readily understand the nature and specific chemistry of the specific LFA.

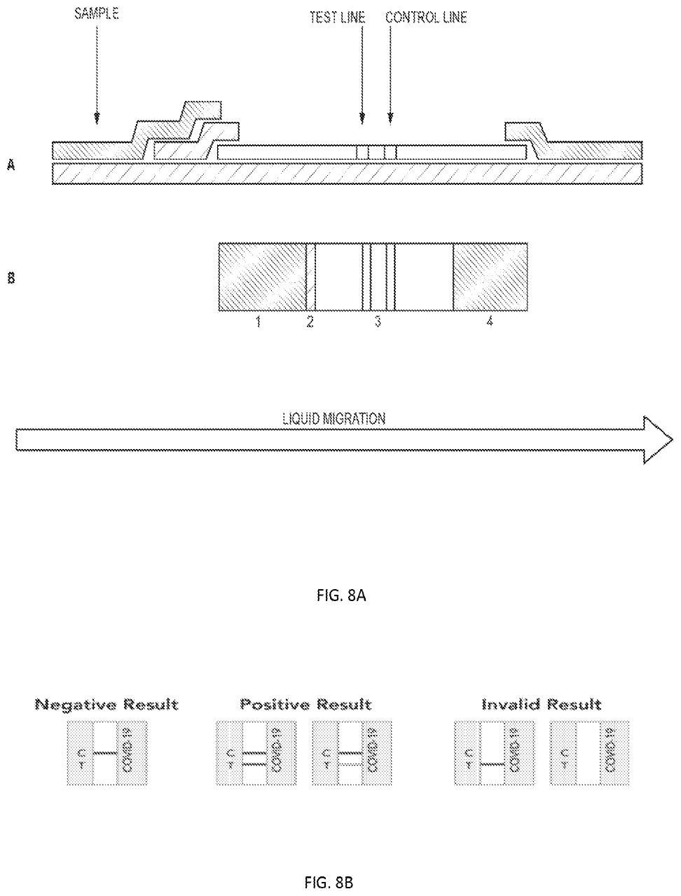

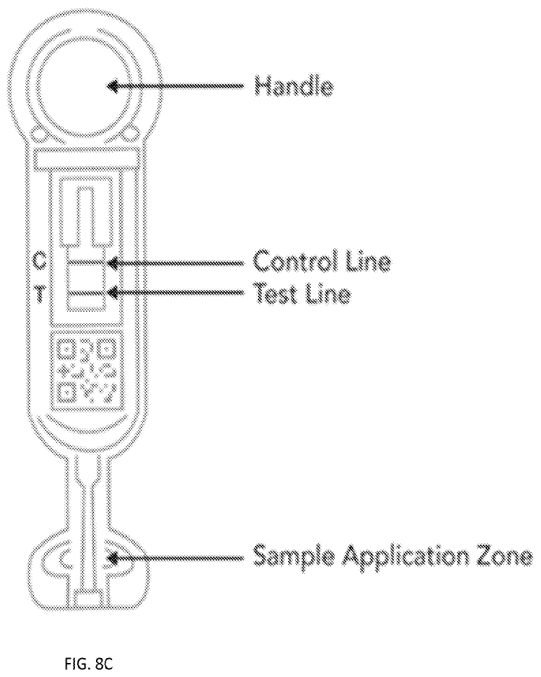

As shown in FIG. 7A Side (A) & Top (B) view of a lateral flow assay (LFA) sample where at (1) the sample (nasal swab, saliva, urine, blood, etc.) is loaded before flowing to the green zone (2), where the target is labeled with a signaling moiety. Through capillary action, the sample will continue flowing until it is immobilized at (3) to form the test line. Excess material is absorbed at (4). FIGS. 8B and 8C disclose the possible outcomes when such a test strip is used, and a typical control line and test line on a common test strip.

When used correctly, these tests are very effective; however self-testing presents challenges for the lay user to interpret. Significant variability is present between devices, making it difficult to tell if the test line you see is negative compared with, for example, faint positive.

To address this challenge, the present disclosure presents, an over-the-counter (OTC) LFA reader that improves the utility of lateral flow assays by enabling rapid and objective readings with a simple, low cost, handheld, and globally-deployable device. The reader disclosed herein reads lateral flow assay tests using TinyML, for example, to accomplish two goals: 1) enable rapid and objective readings of LFAs and 2) streamline digital reporting.

TinyML allows for the software on the reader to be deployed on low-cost hardware that can be widely distributed-which is difficult with existing LFA readers which rely on high-cost/high complexity hardware that cost hundreds to thousands of dollars per unit.

Ultimately, TinyML enables the reader to catch missed positive test results by removing human bias and increasing confidence in lateral flow device testing, reducing user error, and increasing overall result accuracy.

### Example 2

In an example embodiment of the present disclosure, the system and methods disclosed herein maintain a consistent high result accuracy, including up to a 99% overall accuracy, (99% sensitivity, 99% specificity) for model performance when interpreting live-run LFA strips.

The ability to read and compare test data to ensure accuracy is constrained by 2 pieces of hardware: Flash memory and SRAM.

In one example embodiment the Pico4ML Dev kit, which has a 2 MB flash memory is used in the reader 200 to host the .uf2 file and 264 kb of SRAM accommodates the intermediate arrays (among other things) of the model This together with a process and workflow allows for quantifying the model's arena size by first using the interpreter function. See below, where this function was called during setup:

```
  TfLite Status setup_status =
ScreenInit(error_reporter); if (setup_status !=
kTfLiteOk){
  while(1){TF_LITE_REPORT_ERROR(error_reporter, “Set up
  failed\n”);};
}
arena_size = interpreter-
>arena_used_bytes( ); printf(“Arena_Size
Used: %zu \n”, arena_size);
```

In the present example embodiment, the value from the interpreter function during Pico4ML Dev kit boot-up is:
DEV_Module_Init OK Arena_Size Used: 93500
sd_spi_go_low_frequency: Actual frequency: 122070V2-Version Card
R3/R7: 0x1aa R3/R7: 0xff8000
R3/R7: 0xc0ff8000
Card Initialized: High Capacity CardSD card initialized
SDHC/SDXC Card: hc_c_size: 15237Sectors: 15603712
Capacity: 7619 MB
sd_spi_go_high_frequency: Actual frequency: 12500000

With this value available, the appropriate TensorArenaSize may be set. the model uses 93500 bytes of SRAM. By setting the TensorArenaSize to just above that amount 99×1024=101376 bytes, we are able to allocate enough memory to host the model without going over the hardware limits (which also causes the Pico4ML Dev Kit to freeze).

Transforming from Unquantized to Quantized Mode

In an example embodiment, the reader platform interprets a 96×96 image. In the original model design, >99.999% accuracy was achieved with certain embodiments described herein, but the intermediate layer is 96×96×32 at fp32 which requires over 1 MB of memory-which would not fit on the Pico4ML Dev Kit's 264 KB of SRAM. In order to achieve the size requirement for the model, the model needed to go from unquantized to quantized; utilizing full int8 quantization. In essence, instead of treating the tensor values as floating points (float32), those values were correlated to integers (int8).

To optimize quantized data accuracy, the effect of two different quantization strategies was examined using Post-training quantization (PTQ) and Quantization-aware training (QAT).

As described below and depicted in FIG. 9, we compare 3 different models to understand which quantization strategy is best. For reference:
Model 1: 2-layer convolutional network
Model 2: 3-layer convolutional network
Model 3: 4-layer convolutional network

Quantization-aware training (QAT) was found to uniformly outperform post-training quantization (PTQ) and is integrated into the workflow, processor and related algorithms of the present disclosure.

Results with Optimized Workflow

Tested across over 800 real-world test runs, the reader preliminary achieves an overall accuracy of 98.7%.

According to embodiment 1, a diagnostic assay reading device is provided comprising: a housing configured to receive a rapid point of care test (POCT) for testing a sample from a subject and adapted to be of a size that it is handheld; a receiving bay configured for positioning the POCT within the housing; optionally a sample introduction port, an illumination source, a detector, a processor and a data/function interface, wherein the POCT is positioned in a receiving bay within the housing, wherein the illumination source and the detector are positioned in optical communication with the receiving bay and with the test area and, if present, the control area of the POCT, wherein the processor is configured to receive and process images collected by the detector, wherein the processing is provided to reduce noise and interference and interpret received signals captured against set criteria to produce output data, and wherein the data interface is transmits or permit transmission of the output data from the diagnostic assay reading device in the form of a graphical user interface and/or a data link.

According to embodiment 2, the device of embodiment 1 is provided, wherein the illumination source illuminates a portion of the POCT within a wide band visual spectrum.

According to embodiment 3, the device of embodiment 1 or 2 is provided, wherein the illumination source illuminates a portion of the POCT within a narrow-band wavelength for fluorescent excitation.

According to embodiment 4, the device of embodiment 1 to 3 is provided, wherein the illumination source illuminates a portion of the POCT within the wide band visual spectrum and within a narrow-band wavelength for fluorescent excitation.

According to embodiment 5, the device of embodiment 1 to 4 is provided, wherein the detector comprises a wide band CMOS sensor.

According to embodiment 6, the device of embodiment 1 to 5 is provided, wherein the detector comprises a wavelength filtered CMOS sensor.

According to embodiment 7, the device of embodiment 1 to 6 is provided, wherein the detector comprises at least one of a wide band CMOS sensor and a wavelength filtered CMOS sensor.

According to embodiment 8, the device of embodiment 1 to 7 is provided, wherein the processor is configured to capture at least one image within a wide band visual spectrum and one image within a narrow-band wavelength for fluorescent excitation.

According to embodiment 9, the device of embodiment 1 to 8 is provided, wherein the process is configured to capture at least two or more images within a wide band visual spectrum and at least two or more images within a narrow-band wavelength for fluorescent excitation.

According to embodiment 10, the device of embodiment 1 to 9 is provided, wherein the processor is configured to capture and combine multiple reflected signals and stack the captured signals to facilitate noise reduction.

According to embodiment 11, the device of embodiment 1 to 10 is provided, wherein the processor is configured to standardize the captured reflected signals.

According to embodiment 12, the device of embodiment 1 to 11 is provided, wherein the processor is configured to standardize the captured reflected signals and further process the signals using at least one of a machine learning signal processing algorithm and a signal interpretation algorithm to determine whether the captured signal meets a predetermined threshold and record the determination as output data.

According to embodiment 13, the device of embodiment 1 to 12 is provided, wherein the output data is transmitted or transmissible from the device via at least one of a graphical user interface; a QR code generated for each set of output data; a WiFi, Bluetooth, or cellular connection; or via direct network connection.

According to embodiment 14, the device of embodiment 1 to 13 is provided, wherein the output data is stored locally at the processor.

According to embodiment 15, the device of embodiment 1 to 14 is provided, further comprising a sample introduction port, the port defining a fluid pathway extending from an external opening to a sample application area of the POCT.

According to embodiment 16, the device of embodiment 1 to 15 is provided, further comprising a sensor adapted to identify or authenticate that a subject using the device is the same subject that provides the sample.

According to embodiment 17, the device of embodiment 1 to 16 is provided, wherein the processor is adapted to incorporate the identification or authentication of the subject in the output data.

According to embodiment 18, the device of embodiment 1 to 17 is provided, wherein the sensor is adapted to face externally from the device, wherein the sensor comprises a CMOS device and is adapted to capture or log the process of obtaining the sample from the subject and the application of the sample to the POCT.

According to embodiment 19, the device of embodiment 1 to 18 is provided, wherein the output data is comprised in a computer readable code.

According to embodiment 20, the device of embodiment 1 to 19 is provided, wherein the computer readable code comprises a QR code.

According to embodiment 21, the device of embodiment 1 to 20 is provided, wherein the QR code is associated with a key specific to the device such that the output data can be securely associated with the specific device and only read with reference to the key.

According to embodiment 22, the device of embodiment 1 to 21 is provided, wherein the POCT is a lateral flow test strip or a vertical flow test device.

According to embodiment 23, the device of embodiment 1 to 22 is provided, wherein the lateral flow test strip is a nitrocellulose based lateral flow test strip.

According to embodiment 24, a method of conducting a diagnostic assay is provided comprising: introducing a POCT to the device of any of embodiments 1 to 23; introducing a sample containing or suspected of containing an analyte to a sample application area of the POCT; illuminating a test area and/or control area of the POCT using the illumination source; detecting an image signal provided by a label bound or associated with the analyte using the detector in the test and/or control areas; receiving and processing the image signal to reduce noise and background and to determine an attribute of the signal related to signal intensity or power; determining whether a positive or negative signal is present on the POCT associated with the analyte based on the attribute; and generating output data embodying the determination of the positive or negative signal.

According to embodiment 25, the method of embodiment 24 is provided, further comprising a sample introduction port and the sample is introduced to the device after the POCT is introduced to the device.

According to embodiment 26, the method of embodiment 24 or 25 is provided, further comprising confirming, in an automated manner using a sensor of the device, that the subject providing the sample is the subject using the device.

According to embodiment 27, the method of embodiment 24 to 26 is provided, wherein the sensor is adapted to face externally from the device, wherein the sensor comprises a CMOS device and is adapted to capture or log the process of obtaining the sample from the subject and the application of the sample to the POCT.

According to embodiment 28, the method of embodiment 24 to 27 is provided, wherein the output data is comprised in a computer readable code.

According to embodiment 29, the method of embodiment 24 to 28 is provided, wherein the computer readable code comprises a QR code.

According to embodiment 30, the method of embodiment 24 to 29 is provided, wherein the QR code is associated with a key specific to the device such that the output data can be securely associated with the specific device and only read with reference to the key.

According to embodiment 31, the method of embodiment 24 to 30 is provided, further comprising logging one or more of: the time the sample is obtained from the subject, the time the sample is introduced to the POCT; and/or the time that the sample has been in contact with the POCT prior to illumination by the illumination source.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it should be appreciated that throughout the description, discussions utilizing terms such as “processing”, “computing”, “calculating”, “determining”, “displaying”, or the like, refer to the action and processes of a computer system or of a similar electronic computing device that manipulates and transforms data represented as physical (electronic) quantities within the computer system's registers and memories into other data similarly represented as physical quantities within the computer system memories or registers or other such information storage, transmission, or display devices.

The algorithms and displays presented herein are not inherently related to any particular computer or other apparatus. Various general-purpose systems may be used with programs in accordance with the teachings herein, or it may prove convenient to construct more specialized apparatuses to perform the methods of some embodiments. The required structure for a variety of these systems will appear from the description below. In addition, the techniques are not described with reference to any particular programming language, and various embodiments may thus be implemented using a variety of programming languages.

In alternative embodiments, the computer system operates as a stand-alone device or may be connected (e.g., networked) to other computer systems. In a networked deployment, the computer system may operate in the capacity of a server or a client computer system in a client-server network environment, or as a peer computer system in a peer-to-peer (or distributed) network environment.

The computer system may be a server computer (e.g., a database server); a client computer; a personal computer (PC); a tablet, a phablet; a wearable device; a laptop computer; a set-top box (STB); a personal digital assistant (PDA); a cellular telephone; an iPhone; a Blackberry; a processor; a telephone; a web appliance; a network router, switch or bridge; or any computer system capable of executing a set of instructions (sequential or otherwise) that specify actions to be taken by that computer system.

While the computer system-readable medium or computer system-readable storage medium is shown in an exemplary embodiment to be a single medium, the terms “computer system-readable medium” and “computer system-readable storage medium” should be taken to include a single medium or multiple media (e.g., a centralized or distributed database and/or associated caches and servers) that store the one or more sets of instructions. The terms “computer system-readable medium” and “computer system-readable storage medium” shall also be taken to include any medium that is capable of storing, encoding or carrying a set of instructions for execution by the computer system and that causes the computer system to perform any one or more of the methodologies of the presently disclosed technique and innovation.

In general, the routines executed to implement the embodiments of the disclosure, may be implemented as part of an operating system or a specific application, component, program, object, module, or sequence of instructions referred to as “computer programs”. The computer programs typically comprise one or more instructions that are set at various times in various memory and storage devices in a computer, and that, when read and executed by one or more processing units or processors in a computer, cause the computer to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms and that the disclosure applies equally regardless of the particular type of computer system or computer-readable medium used to actually effect the distribution.

Further examples of computer system-readable storage media, computer system-readable media, or computer-readable (storage) media include but are not limited to recordable-type media such as volatile and non-volatile memory devices, floppy and other removable disks, hard disk drives, optical disks (e.g., Compact Disk Read-Only Memory (CD ROMS), Digital Versatile Disks, (DVDs), etc.), and SD cards, among others.

Unless the context clearly requires otherwise, throughout the description and the claims, the words “comprise”, “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein”, “above”, “below”, and words of similar import, when used in this application, shall refer to this application as a whole and not to any particular portions of this application. Where the context permits, words in the above Detailed Description using the singular or plural number may also include the plural or singular number respectively.

The teachings of the disclosure provided herein can be applied to other systems, not only to the system described above. The elements and acts of the various embodiments described above can be combined to provide further embodiments.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

1. A diagnostic assay reading device comprising:
a housing configured to receive a rapid point of care test (POCT) or over-the-counter (OTC) test for testing a sample from a subject and adapted to be of a size that it is handheld;
a receiving bay configured for positioning the POCT or OTC test within the housing; optionally a sample introduction port, an illumination source, a detector, a processor and a data/function interface, wherein the POCT or OTC test is positioned in a receiving bay within the housing,
wherein the illumination source and the detector are positioned in optical communication with the receiving bay and with the test area and, if present, the control area of the POCT or OTC test,
wherein the processor is configured to receive and process images collected by the detector, wherein the processing is provided to reduce noise and interference and interpret received signals captured against set criteria to produce output data, and
wherein the data interface is configured to transmit or permit transmission of the output data from the diagnostic assay reading device in the form of a graphical user interface and/or a data link.

2. The device of claim 1 wherein the illumination source illuminates a portion of the POCT or OTC test:
(a) within a wide band visual spectrum;
(b) within a narrow-band wavelength for fluorescent excitation; or
(c) within the wide band visual spectrum and within a narrow band wavelength for fluorescent excitation.

3-4. (canceled)

5. The device of claim 1 wherein the detector comprises a wide band CMOS sensor, a wavelength filtered CMOS sensor, or at least one of a wide band CMOS sensor and a wavelength filtered CMOS sensor.

6-7. (canceled)

8. The device of claim 1 where the processor is configured to:
(a) capture at least one image within a wide band visual spectrum and one image within a narrow-band wavelength for fluorescent excitation;
(b) capture at least two or more images within a wide band visual spectrum and at least two or more images within a narrow band wavelength for fluorescent excitation;
(c) capture and combine multiple reflected signals and stack the captured signals to facilitate noise reduction, and optionally standardize the captured reflected signals or standardize the captured reflected signals and further process the signals using at least one of a machine learning signal processing algorithm and a signal interpretation algorithm to determine whether the captured signal meets a predetermined threshold and record the determination as output data.

9-12. (canceled)

13. The device of claim 1 wherein the output data is (a) transmitted or transmissible from the device via at least one of a graphical user interface; a QR code generated for each set of output data; a Wifi, Bluetooth, or cellular connection; or via direct network connection; or (b) stored locally at the processor.

14. (canceled)

15. The device of claim 1 further comprising (a) a sample introduction port, the port defining a fluid pathway extending from an external opening to a sample application area of the POCT or OTC test; or (b) a sensor adapted to identify or authenticate that a subject using the device is the same subject that provides the sample.

16. (canceled)

17. The device of claim 15, wherein the processor is adapted to (a) incorporate the identification or authentication of the subject in the output data; or (b) face externally from the device, wherein the sensor comprises a CMOS device and is adapted to capture or log the process of obtaining the sample from the subject and the application of the sample to the POCT or OTC test.

18. (canceled)

19. The device of claim 1, wherein the output data is comprised in a computer readable code.

20. The device of claim 19, wherein the computer readable code comprises a QR code.

21. The device of claim 20, wherein the QR code is associated with a key specific to the device such that the output data can be securely associated with the specific device and only read with reference to the key.

22. The device of claim 1, wherein the POCT or OTC test is a lateral flow test strip or a vertical flow test device.

23. The device of claim 22, wherein the lateral flow test strip is a nitrocellulose based lateral flow test strip.

24. A method of conducting a diagnostic assay comprising:
introducing a POCT or OTC test to the device of claim 1;
introducing a sample containing or suspected of containing an analyte to a sample application area of the POCT or OTC test;
illuminating a test area and/or control area of the POCT or OTC test using the illumination source;
detecting an image signal provided by a label bound or associated with the analyte using the detector in the test and/or control areas;
receiving and processing the image signal to reduce noise and background and to determine an attribute of the signal related to signal intensity or power;
determining whether a positive or negative signal is present on the POCT or OTC test associated with the analyte based on the attribute; and
generating output data embodying the determination of the positive or negative signal.

25. The method of claim 24, further comprising (a) a sample introduction port and the sample is introduced to the device after the POCT or OTC test is introduced to the device; or (b) confirming, in an automated manner using a sensor of the device, that the subject providing the sample is the subject using the device.

26. (canceled)

27. The method of claim 25, wherein the sensor is adapted to face externally from the device, wherein the sensor comprises a CMOS device and is adapted to capture or log the process of obtaining the sample from the subject and the application of the sample to the POCT or OTC test.

28. The method of claim 24, wherein the output data is comprised in a computer readable code.

29. The method of claim 28, wherein the computer readable code comprises a QR code.

30. The method of claim 29, wherein the QR code is associated with a key specific to the device such that the output data can be securely associated with the specific device and only read with reference to the key.

31. The method of claim 24, further comprising logging one or more of: the time the sample is obtained from the subject, the time the sample is introduced to the POCT or OTC test; and/or the time that the sample has been in contact with the POCT or OTC test prior to illumination by the illumination source.
